(19) United States
(12) Patent Application Publication
(10) Pub. No.: US 2025/0129331 A1
(43) Pub. Date: Apr. 24, 2025

(54) MEDIUM COMPOSITION FOR CULTURE OF EXTRAEMBRYONIC ENDODERM STEM CELLS

(71) Applicants: SEOUL NATIONAL UNIVERSITY R&DB FOUNDATION, Seoul (KR); SPACE F CORPORATION, Hwaseong-si, Gyeonggi-do (KR)

(72) Inventors: Chang-Kyu LEE, Seoul (KR); Jinsol JEONG, Seoul (KR); Kwang-Hwan CHOI, Seoul (KR)

(73) Assignees: SEOUL NATIONAL UNIVERSITY R&DB FOUNDATION, Seoul (KR); SPACE F CORPORATION, Hwaseong-si, Gyeonggi-do (KR)

(21) Appl. No.: 18/694,319

(22) PCT Filed: Jul. 4, 2023
(86) PCT No.: PCT/KR2023/009357

§ 371 Date: Mar. 21, 2024

(30) Foreign Application Priority Data
Feb. 23, 2023 (KR) 10-2023-0024352

(51) Int. Cl. C12N 5/0735

(52) U.S. Cl. CPC C12N 5/0606; C12N 2501/113; C12N 2501/115; C12N 2501/117; C12N 2501/119; C12N 2501/235; C12N 2501/39; C12N 2501/395; C12N 2501/415

(57) ABSTRACT

The present invention relates to a medium composition for culture of extraembryonic endoderm stem cells, and more specifically, to a medium composition for in vitro culture of extraembryonic endoderm stem cells.

### TECHNICAL FIELD

This application claims the benefit of priority based on Korean Patent Application No. 10-2023-0024352 filed on Feb. 23, 2023, the entire contents of which are incorporated herein as part of the present specification.

The present invention relates to a medium composition for culture of extraembryonic endoderm stem cells, and more specifically, to a medium composition for in vitro culture of extraembryonic endoderm stem cells.

### BACKGROUND ART

Extraembryonic endoderm (XEN) stem cells (hereinafter referred to as XEN cells) are derived from primitive endoderm (PrE/hypoblast) isolated from the inner cell mass (ICM) within the blastocyst during lineage differentiation in the process of embryonic development. Since XEN cells represent the primitive endoderm lineage, they may provide an opportunity to elucidate the mechanisms underlying developmental processes associated with the primitive endoderm lineage. The first reported XEN cells were reproducibly derived from mouse blastocysts and proliferated in vitro without senescence. Self-renewing XEN cells were characterized by the expression of primitive endoderm lineage-related markers and differentiation ability into the extraembryonic endoderm lineage upon chimera formation. Since then, alternative methods of inducing XEN cells have been discovered as follows:

(1) Separation from trophoblast stem cells (TSCs) in seeded post-implantation embryos, (2) overexpression of Gata4/6, a primitive endoderm marker, (3) directed differentiation using signaling molecules, (4) reprogramming of somatic cells that undergo a XEN-like intermediate process.

Establishment of XEN cells through these methods was mainly studied in mice, followed by studies in rats, humans, pigs, and the like.

Across species, XEN cells were cultured in a fetal bovine serum (FBS)-containing medium supplemented with fibroblast growth factor 4 (FGF4) or leukemia inhibitory factor (LIF) commonly used for culture of trophoblast stem cells (TSCs) and embryonic stem cells (ESCs), respectively. Reportedly, FGF/MAP kinase signaling was critically involved in the specification of primitive endoderm lineages, and LIF/STAT signaling promoted XEN cell proliferation in mice. However, these culture conditions caused defects such as variations among batches and heterogeneous cell populations due to the use of serum-containing media. For example, variation in properties between XEN cell lines and coexistence of trophoblast stem cells (TSCs) or embryonic stem cells (ESCs) in cultures of XEN cells have been reported.

Recently, studies have been attempted to establish a chemically-defined culture system capable of providing reproducible and homogeneous XEN cells to define a reliable developmental mechanism. Consequently, signaling pathways other than fibroblast growth factor (FGF) and leukemia inhibitory factor (LIF) for their maintenance have been identified. For example, a combination of fibroblast growth factor 4 (FGF4), CHIR99021 and platelet-derived growth factor (PDGF-AA) and a combination of leukemia inhibitory factor (LIF), CHIR99021 and activin A were used to derive XEN cells from mouse blastocysts, or naive ESCs of mice or humans, respectively. CHIR99021 and activin A induced cells with primitive endoderm characteristics, and other factors promoted the proliferation of these cells.

Several studies have also been conducted using serum-free conditions supplemented with growth factors or signaling molecules for culture of XEN cells in pigs. However, when compared to the fetal bovine serum-based medium, the knock-out serum replacement (KSR)-based medium to which FGF4 was added showed senescence of porcine XEN cells, and the N2B27-based medium containing fibroblast growth factor 2 (FGF2) and leukemia inhibitory factor (LIF) prolonged the doubling time. This indicates that these culture conditions impaired the self-renewing ability of porcine XEN cells due to inappropriate extrinsic cues compared to serum-containing media. Thus, this demonstrates that porcine XEN cells require the establishment of new culture conditions containing additional growth factors or signaling molecules.

Under these backgrounds, as a result of studying the medium composition for culture of XEN cells from various angles, the present inventors have confirmed a culture system of porcine XEN cells that shows consistent and efficient maintenance through optimization of medium composition, and have completed the present invention.

### PRIOR ART DOCUMENTS

### Patent Documents

(Patent Document 1) US 2021/0227809 A1 (Jul. 29, 2021)
(Patent Document 2) US 2021/0163992 A1 (Jun. 3, 2021)
(Patent Document 3) U.S. Pat. No. 9,388,388 B2 (Jul. 12, 2016)
(Patent Document 4) US 2008/0182328 A1 (Jul. 31, 2008)

### Non-Patent Documents

(Non-Patent Document 1) Kunath T, Arnaud D, Uy G D, Okamoto I, Chureau C, Yamanaka Y, et al. Imprinted X-inactivation in extra-embryonic endoderm cell lines from mouse blastocysts. Development. 2005; 132 (7): 1649-61.
(Non-Patent Document 2) Niakan K K, Schrode N, Cho L T, Hadjantonakis A K. Derivation of extraembryonic endoderm stem (XEN) cells from mouse embryos and embryonic stem cells. Nat Protoc. 2013; 8 (6): 1028-41.
(Non-Patent Document 3) Park C H, Jeoung Y H, Uh K J, Park K E, Bridge J, Powell A, et al. Extraembryonic Endoderm (XEN) Cells Capable of Contributing to Embryonic Chimeras Established from Pig Embryos. Stem Cell Reports. 2021; 16 (1): 212-23.
(Non-Patent Document 4) Shen Q Y, Yu S, Zhang Y, Zhou Z, Zhu Z S, Pan Q, et al. Characterization of porcine extraembryonic endoderm cells. Cell Prolif. 2019; 52 (3): e12591.
(Non-Patent Document 5) Li Y, Wu S, Yu Y, Zhang H, Wei R, Lv J, et al. Derivation of porcine extraembryonic endoderm-like cells from blastocysts. Cell Prolif. 2020; 53 (4): e12782.

### DISCLOSURE

### Technical Problem

It is an object of the present invention to provide a medium composition for culture of XEN cells that shows consistent and efficient maintenance through optimization of medium composition.

### Technical Solution

In order to achieve the above object, the present invention provides a medium composition for culture of stem cells, comprising a fibroblast growth factor, a leukemia inhibitory factor, a WNT signaling activator and a B27 supplement.

According to an embodiment of the present invention, the fibroblast growth factor may be of the FGF family.

According to an embodiment of the present invention, the FGF family may be any one of FGF 1, FGF 2, FGF 4, FGF 7, FGF 10 and FGF 18.

According to an embodiment of the present invention, the WNT signaling activator may be any one of CHIR99021, BIO, WNT agonist 1 and R-spondin.

According to an embodiment of the present invention, the B27 supplement may comprise at least one of corticosterone, progesterone and T3 hormone.

According to an embodiment of the present invention, the content of the fibroblast growth factor may be 10 ng/ml to 100 ng/ml.

According to an embodiment of the present invention, the content of the leukemia inhibitory factor may be 0.1 ng/ml to 20 ng/ml.

According to an embodiment of the present invention, the content of the CHIR99021 may be 0.5 μM to 4.5 μM.

According to an embodiment of the present invention, the stem cells may be extraembryonic endoderm (XEN) stem cells.

In addition, the present invention provides a method of culturing stem cells using the medium composition according to an embodiment.

### Advantageous Effects

The medium composition for culture of stem cells according to the present invention may provide consistent and efficient culture.

The medium composition for culture of stem cells according to the present invention may be applied to mass culture, homogeneous culture, differentiation studies, and molecular biological analysis.

The medium composition for culture of stem cells according to the present invention may be applied to in vitro culture of XEN cells.

The medium composition for culture of stem cells according to the present invention may be applied to provide an alternative cell source for embryonic and extraembryonic endoderm lineages.

The medium composition for culture of stem cells according to the present invention is applied to cell culture modeling representing the primitive endoderm and its derived lineages in mammals, including pigs, and may be used as a useful in vitro tool for in-depth studies of embryology, including lineage segregation, embryo patterning and germ cell differentiation.

The medium composition for culture of stem cells according to the present invention may also be used in livestock other than pigs.

### DESCRIPTION OF DRAWINGS

FIG. 1 shows the results of observing variation in fetal bovine serum-derived characteristics in porcine XEN cells. AP staining represents the results analyzed in six XEN cell groups (scale bar=200 μm).

FIG. 2 shows serum-free culture conditions for culture of porcine XEN cells. In FIG. 2a shows the addition of signaling molecules, and FIG. 2b shows the conditions of medium composition using knockout serum replacements (KSR) and B27 supplements (scale bar=200 μm).

FIG. 3 shows optimized conditions for culture of porcine XEN cells. In FIGS. 3a, 3b and 3c show the results of observing their changes while withdrawing each signaling molecule or supplement for XEN cells.

FIG. 4 shows the results of confirming the optimal concentration of essential factors for porcine XEN cells.

FIG. 5 shows the results of characterizing porcine XEN cells.

### BEST MODE

Hereinafter, the present invention will be described in more detail.

Unless defined otherwise, all technical terms used in the present invention have the same meaning as commonly understood by a person having ordinary knowledge in the relevant field of the present invention. In addition, preferred methods or samples are described in the present specification, but similar or equivalent ones are also included in the scope of the present invention. The contents of all publications described by reference in the present specification are incorporated in the present specification by reference in their entirety.

In describing and claiming particular features of the present disclosure, the following terms will be used in accordance with the definitions set forth below unless otherwise specified.

It should be understood that although some aspects in the present specification are described with the term “comprising,” other similar aspects described in the light of “consisting of” and/or “consisting essentially of” are also provided.

In the present invention, the term “extraembryonic endoderm (XEN) stem cells (hereinafter referred to as XEN cells)” refer to cells derived from primitive endoderm (PrE/hypoblast) isolated from the inner cell mass (ICM) within the blastocyst during lineage differentiation in the process of embryonic development. Since XEN cells represent the primitive endoderm lineage, they may provide an opportunity to elucidate the mechanisms underlying developmental processes associated with the primitive endoderm lineage.

In the present invention, the term “medium,” “culture medium,” “medium for culture,” “medium composition” or “composition for culture” refers to a culture solution containing nutrients capable of supporting the growth and survival of stem cells in in vitro culture conditions, and may be used interchangeably without being distinguished in the present specification.

In the present invention, the term “serum-free medium” or “serum-free condition” refers to a medium or condition that does not contain serum or contains serum only in an amount less than effective enough to exert its biological and physiological function in a culture environment. Serum collectively refers to serum commonly included in cell culture, such as fetal bovine serum (FBS), bovine calf serum (BCS), dialyzed fetal bovine serum and newborn calf serum (NCS). Serum contains a wide range of high-molecular proteins, low-molecular nutrients, carriers for insoluble substances, hormones, and the like, and is often added to the basal medium.

In the present invention, the term “sub-culture” refers to a method of continuously culturing cells from generation to generation while periodically transferring a portion of the cells to a new culture vessel and then changing the culture medium, in order to continuously culture the cells for a long period of time in a healthy state. The term “passage” refers to growth into pluripotent stem cells from the initial seed culture in a culture vessel to the time of vigorous cell growth (confluence) in the same culture vessel. It is used as a method to increase the number of healthy cells because growth nutrients are consumed or contaminants build up and cells naturally die when a certain period of time elapses while the number of cells increases in a culture vessel with a limited space, and usually, replacing the medium (culture vessel) once or culturing by dividing into cell groups is called 1 passage. Methods of sub-culture may use methods known in the art without limitation, but may be preferably performed by mechanical separation or enzymatic separation.

XEN cells have been studied in a variety of species, but most have used media used for culture of trophoblast stem cells (TSCs) and embryonic stem cells (ESCs). The culture system of these media was more suitable for the maintenance of embryonic stem cells (ESCs) or trophoblast stem cells (TSCs) rather than XEN cells.

In addition, most studies have used fetal bovine serum-containing media, which are accompanied by serum-derived defects such as variations among batches and heterogeneous cell culture.

However, various approaches have been attempted to culture XEN cells using a serum-free medium in pigs, but these conditions showed inefficiency during maintenance when compared to a medium containing serum. As an example, porcine XEN cells exhibited inconsistent culture in serum-based media and exhibited lower proliferation rates in the chemically defined culture conditions containing FGF2 and/or LIF compared to serum-based media.

The present inventors confirmed that porcine XEN cells have problems such as variations among cell lines and limited proliferative ability in each of the fetal bovine serum-based medium and the KSR-based medium supplemented with FGF2 and LIF. These results indicate that the above external conditions including the medium composition are insufficient to stably maintain and proliferate porcine XEN cells. Thus, as a result of continuing research in various ways to develop a medium composition for culture of XEN cells, the present inventors have discovered additional XEN cell growth promoting factors and have developed a medium composition for culture of XEN cells that shows consistent and efficient maintenance.

In an embodiment of the present invention, the medium composition for culture of stem cells may comprise a fibroblast growth factor, a leukemia inhibitory factor and a GSK-3 inhibitor.

The fibroblast growth factor refers to a factor capable of promoting the growth of stem cells and various cells, and is known to regulate a wide range of biological functions such as cell proliferation, survival and differentiation.

In an embodiment of the present invention, the fibroblast growth factor may be of the FGF family.

The FGF family may be factors involved in cell proliferation function. Specifically, the FGF family may be any one of FGF 1, FGF 2, FGF 4, FGF 7, FGF 10 and FGF 18, but is not limited to these examples.

In an embodiment of the present invention, the content of the fibroblast growth factor included in the medium composition for culture of stem cells may be 10 ng/ml to 100 ng/ml. More specifically, the content of the fibroblast growth factor included in the medium composition for culture of stem cells may be 10 ng/ml or more, 11 ng/ml or more, 12 ng/ml or more, 13 ng/ml or more, 14 ng/ml or more, 15 ng/ml or more, 16 ng/ml or more, 17 ng/ml or more, 18 ng/ml or more, 19 ng/ml or more, 20 ng/ml or more, 21 ng/ml or more, 22 ng/ml or more, 23 ng/ml or more, 24 ng/ml or more, 25 ng/ml or more, 26 ng/ml or more, 27 ng/ml or more, 28 ng/ml or more, 29 ng/ml or more, or 30 ng/ml or more, or may be 100 ng/ml or less, 99 ng/ml or less, 98 ng/ml or less, 97 ng/ml or less, 96 ng/ml or less, 95 ng/ml or less, 94 ng/ml or less, 93 ng/ml or less, 92 ng/ml or less, 91 ng/ml or less, 90 ng/ml or less, 89 ng/ml or less, 88 ng/ml or less, 87 ng/ml or less, 86 ng/ml or less, 85 ng/ml or less, 84 ng/ml or less, 83 ng/ml or less, 82 ng/ml or less, 81 ng/ml or less, or 80 ng/ml or less.

When the content of the fibroblast growth factor (FGF) is less than 10 ng/ml, the growth of the stem cell population is inhibited and the expression of differentiation markers is increased, and thus, it is difficult to maintain the undifferentiated state of stem cells, and when it is more than 100 ng/ml, additional effects according to the increase in concentration cannot be expected.

The leukemia inhibitory factor (LIF) refers to an interleukin 6 (IL-6)-type cytokine involved in various biological activities and affecting different cell types. LIF is an important signaling molecule and is known to play an important role in diseases associated with unwanted cell proliferation, such as various types of tumors.

In an embodiment of the present invention, the content of the leukemia inhibitory factor included in the medium composition for culture of stem cells may be 0.1 ng/ml to 20 ng/ml. More specifically, the content of the fibroblast growth factor included in the medium composition for culture of stem cells may be 0.1 ng/ml or more, 0.2 ng/ml or more, 0.3 ng/ml or more, 0.4 ng/ml or more, 0.5 ng/ml or more, 0.6 ng/ml or more, 0.7 ng/ml or more, 0.8 ng/ml or more, 0.9 ng/ml or more, 1.0 ng/ml or more, 2.0 ng/ml or more, 3.0 ng/ml or more, 4.0 ng/ml or more, or 5.0 ng/ml or more, or may be 20 ng/ml or less, 19 ng/ml or less, 18 ng/ml or less, 17 ng/ml or less, 16 ng/ml or less, 15 ng/ml or less, 14 ng/ml or less, 13 ng/ml or less, 12 ng/ml or less, 11 ng/ml or less, or 10 ng/ml or less.

When the content of the leukemia inhibitory factor (LIF) is less than 0.1 ng/ml, the growth of the stem cell population is inhibited and the expression of differentiation markers is increased, and thus, it is difficult to maintain the undifferentiated state of stem cells, and when it is more than 20 ng/ml, additional effects according to the increase in concentration cannot be expected.

The WNT signaling activator is a regulator for signaling activity initiated by the binding of Wnt signaling proteins to receptors on the cell surface, and may regulate gene expression associated with cell proliferation.

The WNT signaling activator may include a GSK-3 inhibitor, a WNT agonist, and a WNT modulator.

The GSK-3 inhibitor is an inhibitor of glycogen synthase kinase-3 (GSK-3) activity, and refers to those that inhibits the activity of GSK-3, which is a proline-directed serine/threonine kinase that performs phosphorylation of multiple protein substrates.

In an embodiment of the present invention, the GSK-3 inhibitor may be CHIR99021 (also referred to as CT99021), BIO, and the like, but is not limited to these examples.

In an embodiment of the present invention, the WNT agonist may include WNT agonist 1 and the like, but is not limited to this example.

In an embodiment of the present invention, the WNT modulator may include R-spondin and the like, but is not limited to this example.

In an embodiment of the present invention, the content of the CHIR99021, which is one of the GSK-3 inhibitors, may be 0.5 μM to 4.5 M. More specifically, the content of the CHIR99021 may be 0.5 μM or more, 1.0 μM or more, 1.5 μM or more, 2.0 μM or more, or 2.5 μM or more, or may be 4.5 μM or less, 4.0 μM or less, 3.5 μM or less, or 3.0 μM or less.

When the content of the CHIR99021 is less than 0.5 μM, the growth of the stem cell population is inhibited and the expression of differentiation markers is increased, and thus, it is difficult to maintain the undifferentiated state of stem cells, and when it is more than 4.5 μM, additional effects according to the increase in concentration cannot be expected.

In an embodiment of the present invention, the content of the BIO, which is one of the GSK-3 inhibitors, may be 0.005 μM to 5 μM. More specifically, the content of the BIO may be 0.005 μM or more, 0.01 μM or more, 0.02 μM or more, 0.03 μM or more, 0.04 μM or more, 0.05 μM or more, 0.06 μM or more, 0.07 μM or more, 0.08 μM or more, 0.09 μM or more, or 0.1 μM or more, or may be 5 μM or less, 4.5 μM or less, 4.0 μM or less, 3.5 μM or less, or 3.0 μM or less.

When the content of the BIO is less than 0.005 μM, the growth of the stem cell population is inhibited and the expression of differentiation markers is increased, and thus, it is difficult to maintain the undifferentiated state of stem cells, and when it is more than 5.0 μM, additional effects according to the increase in concentration cannot be expected.

In an embodiment of the present invention, the content of the WNT agonist 1, which is one of the WNT agonists, may be 0.7 μM to 20 μM. More specifically, the content of the WNT agonist 1 may be 0.7 μM or more, 1.0 μM or more, 2.0 μM or more, 3.0 μM or more, 4.0 μM or more, or 5.0 μM or more, or may be 20 μM or less, 18 μM or less, 16 μM or less, 15 μM or less, 13 μM or less, 11 μM or less, or 9 μM or less.

When the content of the WNT agonist 1 is less than 0.7 UM, the growth of the stem cell population is inhibited and the expression of differentiation markers is increased, and thus, it is difficult to maintain the undifferentiated state of stem cells, and when it is more than 20 μM, additional effects according to the increase in concentration cannot be expected.

In an embodiment of the present invention, the content of R-spondin, which is one of the WNT modulators, may be 1 ng/ml to 100 ng/ml. More specifically, the content of the R-spondin may be 1 ng/ml or more, 5 ng/ml or more, 10 ng/mL or more, 15 ng/ml or more, 20 ng/ml or more, 25 ng/mL or more, or 30 ng/ml or more, or may be 100 ng/ml or less, 90 ng/ml or less, 80 ng/ml or less, 70 ng/ml or less, 60 ng/ml or less, or 50 ng/ml or less.

When the content of the R-spondin is less than 1 ng/ml, the growth of the stem cell population is inhibited and the expression of differentiation markers is increased, and thus, it is difficult to maintain the undifferentiated state of stem cells, and when it is more than 100 ng/ml, additional effects according to the increase in concentration cannot be expected.

It was confirmed that the medium composition for culture of stem cells comprising a B27 supplement according to an embodiment of the present invention showed effective cell proliferation by using the B27 supplement related to lipid metabolism.

The B27 supplement may comprise at least one of corticosterone, progesterone and T3 hormone, but is not limited to these examples.

The medium composition for culture of stem cells comprising a B27 supplement according to an embodiment of the present invention showed the effect of providing a homogeneous culture of porcine XEN cells at a rapid proliferation rate without loss of stemness.

The basal medium of the medium composition for culture of stem cells comprising a fibroblast growth factor, a leukemia inhibitory factor and a GSK-3 inhibitor according to an embodiment of the present invention may optionally be selected from conventional media appropriately used for culture of stem cells in the field. In addition, the culture conditions may also be optionally selected from appropriate conditions used in the field. That is, the medium and culture conditions may be selected according to the type of cells to be cultured. The medium to be used for culture is a cell culture minimum medium (CCMM), which generally contains a carbon source, a nitrogen source and trace element components.

The cell culture minimal medium that may be used in the present invention includes, but is not necessarily limited to, DMEM (Dulbecco's Modified Eagle's Medium), MEM (Minimal Essential Medium), BME (Basal Medium Eagle), RPMI1640, F-10, F-12, aMEM (a-modified Minimum Essential Media), GMEM (Glasgow's Minimal Essential Medium), IMDM (Iscove's Modified Dulbecco's Medium), and the like. In the present invention, DMEM/F-12 medium used for cell culture in the field is used as the basal medium.

In the medium composition for culture according to the present invention, it is preferable to additionally use antibiotics, antifungal agents, and/or substances commonly used in the field to prevent the growth of mycoplasma, in order to prevent infection by bacteria, fungi, and the like. All antibiotics commonly used for cell culture, such as penicillin-streptomycin, may be used as the antibiotics, and commonly used substances, such as amphotericin B as the antifungal agent, and gentamicin, ciprofloxacin, azithromycin or the like as a mycoplasma inhibitor, may be used, but are not limited thereto. In addition, a commercially available antibiotic-antimycotic (AA) (Gibco) may be used.

The medium composition for culture according to the present invention may comprise fetal bovine serum (FBS). The content of the FBS included in the medium is 5% to 20%. The medium composition for culture of stem cells according to an embodiment of the present invention may comprise 5%, 10%, 15% or 20% FBS, and preferably, comprise 10% FBS. In this case, % refers to % v/v.

The medium composition for culture according to the present invention may comprise 1% glutamax (or glutamine) and/or 0.1 mM β-mercaptoethanol.

The medium composition for culture according to the present invention may comprise a knockout serum replacement (KSR). The content of the KSR included in the medium is 5% to 15%. The medium composition for culture of stem cells according to an embodiment of the present invention may comprise 5%, 10% or 15% of KSR. In this case, % refers to % v/v.

The medium composition for culture according to the present invention may be used for culture of stem cells. In this case, the stem cells may be extraembryonic endoderm stem cells.

The medium composition for culture according to the present invention may be applied to in vitro culture of extraembryonic endoderm stem cells.

The medium composition for culture according to the present invention may be provided as an alternative cell source for embryonic and extraembryonic endoderm lineages.

The extraembryonic endoderm stem cells according to the present invention may be porcine XEN cells, but are not limited to this example, and may also be applied to livestock such as cattle, horses, sheep and goats.

The medium composition for culture according to the present invention is applied to cell culture modeling representing the primitive endoderm and its derived lineages in mammals, and may be used as a useful in vitro tool for in-depth studies of embryology, including lineage segregation, embryo patterning and germ cell differentiation.

There is provided a method of culturing stem cells using the medium composition for culture of stem cells according to an embodiment of the present invention.

The culture method may comprise the step of passaging the stem cells. The method may maintain the stem cells in an undifferentiated state, and specifically, may maintain the stem cells in an undifferentiated state during or after sub-culture. The method may maintain stem cells without loss of stemness even after, for example, 3 to 10 or more passages.

Unless otherwise indicated, all numbers used in the present specification and claims, whether recited or not, are to be understood as being modifiable by the term “about” in all instances. It is also to be understood that the precise numbers used in the specification and claims form additional embodiments of the present disclosure. Efforts have been made to ensure the accuracy of the numerical values disclosed in the examples. However, all measured values may inherently include certain error values generated from the standard deviations measured in their respective measurement techniques.

### BEST MODE

Hereinafter, the present invention will be described in more detail through examples. It will be apparent to those skilled in the art that these examples are only for illustrating the present invention in more detail and the scope of the present invention is not limited to these examples in accordance with the gist of the present invention.

### Example 1. Culture of XEN Cells

### Example 1-1. Culture for Maintenance of Porcine XEN Cells

The management and experimental use of pigs and mice was approved by the Institutional Animal Care and Use Committee at Seoul National University (Approval No. SNU-191025-4-4). The ovaries used were donated from a domestic slaughterhouse (Anyang-si, Gyeonggi-do, Republic of Korea) and used for research. Pregnant ICR mice were purchased from Samtaco Bio Korea (Republic of Korea). Mice were managed according to the standard protocol of the Institute of Laboratory Animal Resources at Seoul National University.

Porcine XEN cells were cultured under various conditions. Each of six fetal bovine serum (FBS) (three different batches purchased from Hyclone, Biowest, TCB and Genedepot) was applied to maintain porcine XEN cells. In addition, several signaling molecules and supplements were added to the basal medium supplemented with 15% (v/v), 10% (v/v) or 5% (v/v) knockout serum replacement (KSR) (Gibco; Gaithersburg, MD, USA) and tested. Factors tested were fibroblast growth factor 2 (FGF2) (R&D Systems; Minneapolis, MN, USA), human recombinant leukemia inhibitory factor (hrLIF) (Millipore; MA, USA), 5 ng/ml activin A (Activin A; R&D Systems), CHIR99021 (Cayman chemical; Ann Arbor, MI, USA), which is a GSK-3B inhibitor, and 1×B27 supplement (Gibco). The basal medium was a DMEM/F12 based medium, which consisted of 1× Glutamax, 0.1 mM β-mercaptoethanol and 1× antibiotic-antimycotic (all of which are manufactured by Gibco). The medium was changed every 24 hours and cultured under conditions of 5% CO₂, 5% O₂ and 38° C.

### Example 1-2. Derivation and Culture of Porcine XEN Cells

The hatched porcine blastocysts were seeded on mitotically inactivated mouse embryonic fibroblasts using XEN cell derivation medium at 38° C. under humidified conditions including 5% CO₂ and 5% O₂. The XEN cell derivation medium is a basal medium, which contains 15% (v/v) knockout serum replacement (KSR), 1×MEM non-essential amino acids, 0.1% (v/v) LC (all of which are manufactured by Gibco), 10 ng/ml human recombinant leukemia inhibitory factor (hrLIF) (Millipore) and 10 ng/mL fibroblast growth factor 2 (FGF2) (R&D Systems). About 14 days after seeding, primary colonies of porcine XEN cells were mechanically dissociated using a pulled glass-pipette and transferred to new feeder cells for sub-culture.

Porcine XEN cells were cultured in XEN cell culture medium containing the following factors in the basal medium: 5% (v/v) knockout serum replacement (KSR) (Gibco), 20 ng/ml fibroblast growth factor 2 (FGF2) (R&D Systems), 10 ng/ml human recombinant leukemia inhibitory factor (hrLIF) (Millipore), 1.5 μM CHIR99021 (Cayman chemical), and 1×B27 supplement (Gibco). Porcine XEN cells were passaged every 4 days. 24 hours before sub-culture, the porcine XEN cells were cultured in XEN cell culture medium containing 10 μM Y-27632 (Santa Cruz Biotechnology; Dallas, TX, USA). Next, the expanded colonies were dissociated into small clumps using TrypLE Express (Gibco). The clumps were transferred to new feeder cells and cultured in XEN cell culture medium containing 10 μM Y-27632 for 24 hours. Next, the adherent clumps were cultured in XEN cell culture medium lacking Y-27632. The medium was changed every 24 hours and incubated under conditions of 5% CO₂, 5% O₂ and 38° C.

### Example 1-3. Formation of XEN-Derived Spheroids for Differentiation Induction into Primitive Endoderm Lineage

To evaluate the in vitro differentiation ability, spheroids were generated from porcine XEN cells. The cultured porcine XEN cells were dissociated into small clumps using TrypLE Express and cultured on an ultralow-attachment plate (Sigma-Aldrich; St. Louis, MO, USA) for 5 days using the following medium: DMEM (Welgene; Gyeongsan-si, Gyeongsangbuk-do, Republic of Korea) containing 10% (v/v) fetal bovine serum (FBS) (Genedepot; Katy, TX, USA), 1× Glutamax, 0.1 mM β-mercaptoethanol, 1× antibiotic-antimycotic and 10 μM Y-27632 (only day 1). After suspension culture, the dissociated cells aggregated to form XEN-derived spheroids.

### Example 1-4. Alkaline Phosphatase (AP) Assay

Before staining, all cell samples were pre-cultured at 4° C. for 10 minutes and fixed with 4% paraformaldehyde for 15 minutes. After washing with Dulbecco's Phosphate Buffered Saline (DPBS) (Welgene), the fixed cells were stained with a buffer solution containing nitro blue tetrazolium chloride and 5-bromo-4-chloro-3-indolyl phosphate toluidine salt stock solution (Roche; Basel, Switzerland) at room temperature for 30 minutes. Stained cells were observed under an inverted microscope.

### Example 1-5. Immunocytochemical (ICC) Assay

For immunostaining, the cells were fixed in 4% (w/v) paraformaldehyde. After washing with Dulbecco's Phosphate Buffered Saline (DPBS), the fixed cells were treated with 0.2% Triton-X100 (Sigma-Aldrich) at 4° C. for 2 hours. The cells were then treated with Dulbecco's Phosphate Buffered Saline (DPBS) containing 10% (v/v) goat or donkey serum for 1 hour to prevent non-specific binding. Serum-treated cells were cultured with primary antibodies at 4° C. for 24 hours. The primary antibodies used were as follows: rabbit anti-SOX2 (1:200, Millipore; AB5603), rabbit anti-NANOG (1:200, PeproTech; Rocky Hill, NJ, USA; 500-P236), chicken anti-OCT4 (1:100, Abcam; Cambridge, UK; ab134218), goose anti-GATA6 (15 μg/mL; R&D systems; AF1700), goose anti-SOX17 (1:200, R&D systems; AF1924), and rabbit anti-GATA4 (1:200, Abcam; ab84593). After incubation with primary antibodies, the cells were treated with Alexa Fluor-conjugated secondary antibodies at 4° C. for 24 hours. Nuclei were stained with Hoechst 33342 (Molecular Probes; Eugene, OR, USA). Images of stained cells were taken using a TE2000-U inverted fluorescence microscope (Nikon; Tokyo, Japan).

### Example 1-6. Gene Expression Analysis by PCR (qPCR)

Total RNA was extracted using TRIzol reagent (Invitrogen; Carlsbad, USA) and cDNA was synthesized using High-Capacity RNA-to-cDNA Kit (Applied Biosystems; Waltham, MA, USA). cDNA was amplified using the primer sets listed in Table 1 below and PowerSYBR® Green PCR Master Mix (Applied Biosystems).

TABLE 1

| [symbol] | [symbol] | [symbol] | [symbol] |
|---|---|---|---|
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |
| [symbol] | [symbol] | [symbol] | [symbol] |
| | [symbol] | | |

[symbol]

indicates data missing or illegible when filed

### Example 1-7. Karyotyping

Cell karyotyping was performed using standard G-banding chromosome and cytogenetic analysis at GenDix Laboratories (www.gendix.com; Republic of Korea).

### Example 2. Screening of Cell Culture Conditions for Maintenance of Porcine XEN Cells

Generally, feeder-conditioned media and fetal bovine serum-containing media were used for XEN cells. FIG. 1 shows the results of confirming variation in fetal bovine serum-derived characteristics in porcine XEN cells. In FIG. 1, AP staining was performed on six XEN cell groups. FIGS. 1A to 1F were cultured with fetal bovine serum from different companies or batches (scale bar=200 μm). When XEN cells were cultured in a serum-containing medium, six fetal bovine serum products obtained from batches various showed variations in characteristics such as AP activity, proliferation, cell-cell adhesion, and spontaneous differentiation in porcine XEN cells, as shown in Table 2 below (FIG. 1). Table 2 below shows variations in characteristics according to fetal bovine serum products in porcine XEN cells.

TABLE 2

| FBS | | Cell-cell | | AP |
|---|---|---|---|---|
| Company/Batch [symbol] | Proliferation | adhesion | Differentiation | staining |
| (A) | [symbol] | [symbol] | [symbol] | [symbol] |
| (B) | [symbol] | [symbol] | [symbol] | [symbol] |
| (C) | [symbol] | [symbol] | [symbol] | [symbol] |
| (D) | [symbol] | [symbol] | [symbol] | [symbol] |
| (E) | [symbol] | [symbol] | [symbol] | [symbol] |
| (F) | [symbol] | [symbol] | [symbol] | [symbol] |

[symbol]

indicates data missing or illegible when filed

a: Each of groups (A) to (F) was cultured with each fetal bovine serum (FBS) from different companies or batches.
b: Scores are relative (+ indicates high value, and − indicates low value).

In particular, it is known that the intensity of AP staining is positively correlated with the proliferative ability of XEN cells. In Table 2 above, group (F) showed the highest AP activity, proliferation rate and cell-cell adhesion, whereas group (E) underwent some degree of differentiation characterized by disappearance of colony boundaries and morphological changes.

This suggests that unknown factors, including growth factors and signaling molecules derived from fetal bovine serum, prevented consistent culture of XEN cells. For this reason, in order to avoid defects caused by the use of serum, an N2B27-based FGF2 or LIF-containing medium, which is commonly used for serum-free conditions of XEN cells, has been conventionally applied. However, the medium for the serum-free conditions of the XEN cells has a problem in that it is less efficient than the fetal bovine serum-based medium. Thus, the present inventors have developed a culture system for porcine XEN cells that is maintained consistently and efficiently through optimization of medium composition.

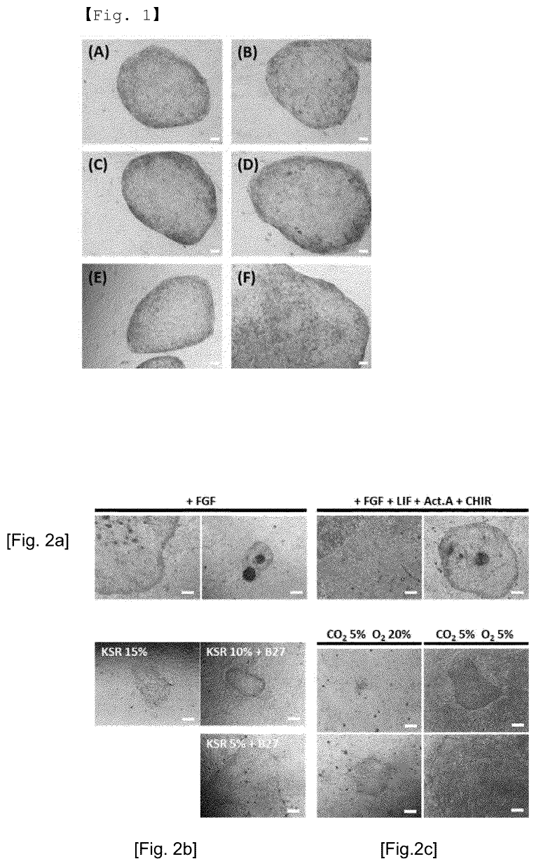

The present inventors performed screening of various factors in a DMEM/F12-based KSR-containing medium, which is commonly used for a serum-free medium in stem cells. As a result, since fibroblast growth factor (FGF) alone was insufficient to proliferate XEN cells, it still showed a prolonged doubling time (FIG. 2a).

FIG. 2 shows the results of serum-free culture conditions for culture of porcine XEN cells. As the serum-free culture conditions for the XEN cells, FIG. 2a shows the results of adding signaling molecules, and FIG. 2b shows the results of the medium composition conditions using KSR and B27 supplements. The left and right images in FIG. 2a show changes before sub-culture and on day 3 after sub-culture, respectively, and FIG. 2b shows changes on day 7 after sub-culture.

In the present invention, leukemia inhibitory factor (LIF), activin A (Act A) and CHIR99021 (CHIR) were sequentially supplemented in the medium. As a result, the medium containing all four factors (FGF2, LIF, Act A and CHIR) showed the fastest growth, indicating that these factors may effectively expand porcine XEN cells (FIG. 2a).

When porcine XEN cells were cultured in a 15% knockout serum replacement (KSR)-based medium supplemented with the aforementioned four factors, lipid droplet-embedded colonies were observed. However, over several passages, the size and number of lipid droplets increased significantly and colony integrity was impaired (FIG. 2b). In order to improve these, the medium composition was tuned through the use of B27 supplement related to lipid metabolism and the modulation of KSR concentration. The 10% KSR-based medium containing the B27 supplement reduced lipid droplets and showed intact colonies with distinct boundaries and compact structures, compared to the 15% KSR-based medium (FIG. 2b). In addition, cell proliferation was greatly promoted when the KSR concentration was lowered from 10% to 5% (FIG. 2b).

### Example 3. Optimization of Culture Conditions of Porcine XEN Cells

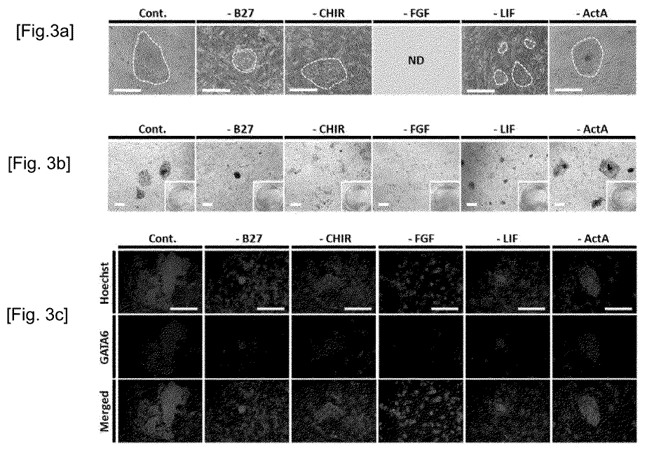

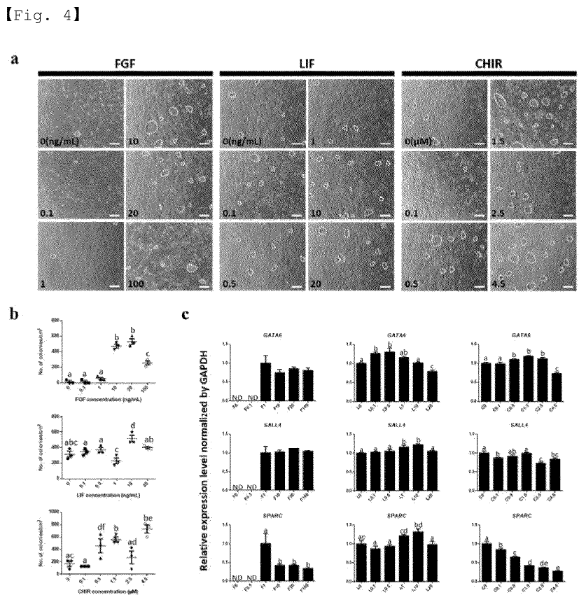

FIG. 3 shows the results of optimized conditions for culture of porcine XEN cells. FIGS. 3a, 3b and 3c show changes in the withdrawal of signaling molecules or supplements for XEN cells. In FIG. 3, the control group is a culture condition containing all of B27 supplement, CHIR, FGF2, LIF and Act A. FIG. 3a shows cell images, and ND indicates not detected. FIG. 3b shows images of AP staining, and FIG. 3c shows immunostaining for XEN cell markers.

In order to optimize the culture system of porcine XEN cells, B27 supplement, CHIR, FGF2, LIF or Act A was withdrawn from the medium during maintenance, and essential growth factors and signaling molecules were identified. Colonies were morphologically analyzed (FIG. 3a). In the absence of B27 supplement or LIF, cell proliferation was inhibited and high-dense small colonies were generated. This pattern was enhanced when FGF2 was withdrawn, and there were no colonies after 4 days. In the absence of CHIR, colony boundaries collapsed and cell density within colonies decreased. In the control group, colony integrity was impaired compared to the condition in which Act A was withdrawn, indicating that a high concentration of Act A rather inhibits the self-renewal of porcine XEN cells and Act A generated in the feeder is sufficient for maintenance. In order to verify the responsiveness to these factors, AP staining and ICC for GATA6, which is a representative XEN cell marker, were performed to support the morphological analysis results (FIGS. 3b and 3c). AP staining and GATA6 expression were evenly positive in the group in which Act A was withdrawn. On the other hand, in the other groups, expression was detected locally within disrupted (Cont., -CHIR and -FGF groups) or aggregated (-B27 and -LIF groups) colonies (“-” refers to the absence, for example, -LIF refers to the absence of LIF). Notably, the absence of LIF induced intense GATA6 expression only in the colony center to result in spontaneous differentiation around the colony edges.

In addition, in order to fine-tune the culture system, porcine XEN cells were cultured under various concentrations of essential factors including FGF2, LIF and CHIR99021.

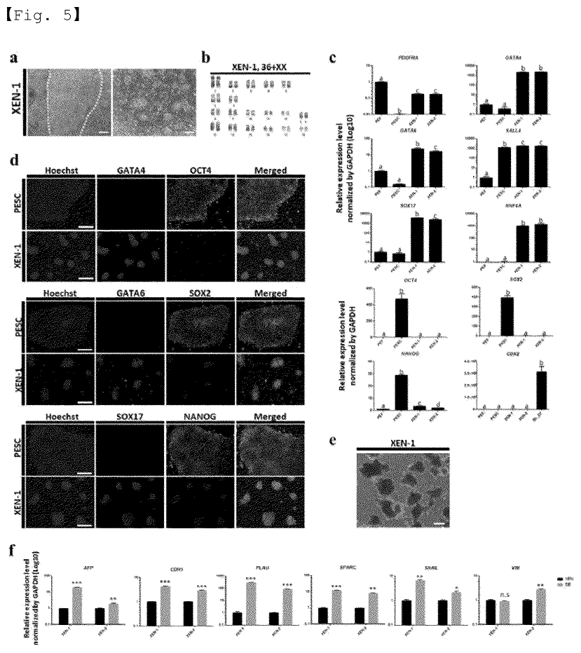

FIG. 4 shows the results of confirming the optimal concentration of essential factors for porcine XEN cells. FIG. 4a shows XEN cells treated with each condition for 7 days, wherein the dashed line represents intact XEN colonies (scale bar=200 μm). FIG. 4b shows the number of XEN colonies per area (cm²), and FIG. 4c shows the qPCR results related to XEN (GATA6 and SALL4) and differentiation markers (SPARC), wherein F is FGF2, L is LIF, C is CHIR99021, and ND indicates not detected.

The lower the concentration of each factor, the smaller the number of XEN colonies tended to be, and in particular, few colonies were detected at low FGF2 concentrations (FIGS. 4a and 4b). In addition, low concentrations of the factors impaired the stemness of XEN along with decreased expression of XEN markers and increased expression of differentiation markers (FIG. 4c). On the other hand, there were no additional effects according to the increase in concentration. Based on these, it was confirmed that the concentration ranges of 10-100 ng/ml for FGF2, 0.1-20 ng/ml for LIF and 0.5-4.5 μM for CHIR99021 were appropriate culture conditions for XEN cell culture.

### Example 4. Characterization of Porcine XEN Cells

FIG. 5 shows the results of characterizing porcine XEN cells. FIG. 5a shows the derivation and maintenance of the XEN-1 cell line from blastocysts, wherein the left and right images show blastocysts maintained on day 13 after seeding and after sub-culture, respectively (scale bar=200 μm). FIG. 5b shows the karyotype of the XEN-1 cell line, and FIG. 5c shows the qPCR results for XEN cell markers, wherein PEF indicates porcine embryonic fibroblasts, PESC indicates porcine embryonic stem cells, and BL indicates blastocysts. FIG. 5d shows immunostaining for the XEN cell markers (scale bar=200 μm). FIG. 5e shows spheroids derived from the XEN-1 cell line on day 5 (scale bar=200 μm). FIG. 5f shows the qPCR results for VE and PE-related markers in XEN-derived spheroids on day 5.

Stable porcine XEN cell lines (XEN-1 and XEN-2) were established from IVF blastocysts with normal karyotypes through the culture conditions optimized in Example 4 above (FIGS. 5a and 5b), and their characteristics as XEN cells were verified. Expression of XEN-specific markers such as PDGFRA, GATA4, GATA6, SALL4, SOX17 and HNF4A was investigated by qPCR (FIG. 5c). Expression levels of these genes in XEN cells were significantly higher than in porcine embryonic stem cells (PESC) or porcine embryonic fibroblasts (PEF). PDGFRA and SALL4 were expressed not only in XEN cells but also in fibroblasts and embryonic stem cells, respectively. In addition, it was confirmed that porcine XEN cells were negative for the Epi markers OCT4, SOX2 and NANOG and the TE marker CDX2. At the protein level, strong expression of GATA4, GATA6 and SOX17 was detected, whereas OCT4, SOX2 and NANOG were absent or faded (FIG. 5d).

In order to evaluate the differentiation ability of porcine XEN cells, we investigated whether the porcine XEN cells could differentiate into the primitive endoderm lineage by forming XEN-derived spheroids in porcine XEN cell lines (FIG. 5e) and inducing spontaneous differentiation. Five days after the formation of XEN-derived spheroids, spheroids were sampled, and gene expression associated with parietal endoderm (AFP, CDH1, PLAU) and visceral endoderm markers (SPARC, SNAIL, VIM) was analyzed by qPCR (FIG. 5f). Their expression levels were significantly higher in the spheroids derived from the porcine XEN cell line than in the undifferentiated porcine XEN cell lines.

1. A medium composition for culture of stem cells, comprising:
a fibroblast growth factor (FGF);
a leukemia inhibitory factor (LIF);
a WNT signaling activator; and
a B27 supplement.

2. The medium composition for culture of stem cells according to claim 1, wherein the fibroblast growth factor is of the FGF family.

3. The medium composition for culture of stem cells according to claim 2, wherein the FGF family is any one of FGF 1, FGF 2, FGF 4, FGF 7, FGF 10 and FGF 18.

4. The medium composition for culture of stem cells according to claim 1, wherein the WNT signaling activator is any one of CHIR99021, BIO, WNT agonist 1 and R-spondin.

5. The medium composition for culture of stem cells according to claim 1, wherein the B27 supplement comprises at least one of corticosterone, progesterone and T3 hormone.

6. The medium composition for culture of stem cells according to claim 1, wherein the content of the fibroblast growth factor is 10 ng/ml to 100 ng/ml.

7. The medium composition for culture of stem cells according to claim 1, wherein the content of the leukemia inhibitory factor is 0.1 ng/ml to 20 ng/ml.

8. The medium composition for culture of stem cells according to claim 4, wherein the content of the CHIR99021 is 0.5 μM to 4.5 μM.

9. The medium composition for culture of stem cells according to claim 1, wherein the stem cells are extraembryonic endoderm (XEN) stem cells.

10. A method of culturing stem cells using a medium composition comprising:
a fibroblast growth factor (FGF);
a leukemia inhibitory factor (LIF);
a WNT signaling activator; and
a B27 supplement.

11. The method according to claim 10, wherein the fibroblast growth factor is of the FGF family.

12. The method according to claim 11, wherein the FGF family is any one of FGF 1, FGF 2, FGF 4, FGF 7, FGF 10 and FGF 18.

13. The method according to claim 10, wherein the WNT signaling activator is any one of CHIR99021, BIO, WNT agonist 1 and R-spondin.

14. The method according to claim 10, wherein the B27 supplement comprises at least one of corticosterone, progesterone and T3 hormone.

15. The method according to claim 10, wherein the content of the fibroblast growth factor is 10 ng/ml to 100 ng/ml.

16. The method according to claim 10, wherein the content of the leukemia inhibitory factor is 0.1 ng/ml to 20 ng/ml.

17. The method according to claim 13, wherein the content of the CHIR99021 is 0.5 μM to 4.5 μM.

18. The method according to claim 10, wherein the stem cells are extraembryonic endoderm (XEN) stem cells.
